(19) United States
(12) Patent Application Publication
(10) Pub. No.: US 2015/0024514 A1
(43) Pub. Date: Jan. 22, 2015

(54) METHOD AND DEVICE FOR DETECTING ANALYTES

(71) Applicant: SECURETEC DETEKTIONS-SYSTEME AG, Brunnthal (DE)

(72) Inventors: Torsten Stadthagen, Muenchen (DE); Frank Schwieger, Taufkirchen (DE); Sebastian Klaus, Neuried (DE)

(73) Assignee: SECURETEC DETEKTIONS-SYSTEME AG, Brunnthal (DE)

(21) Appl. No.: 14/383,149

(22) PCT Filed: Mar. 6, 2013
(86) PCT No.: PCT/EP2013/054499

§ 371 Date: Sep. 5, 2014

(30) Foreign Application Priority Data
Mar. 6, 2012 (EP) 12158247.2

(51) Int. Cl. G01N 33/94

(52) U.S. Cl. CPC G01N 33/948; G01N 33/946
USPC 436/501; 422/70

(57) ABSTRACT

The present invention relates to a method for determining analytes and to a device suitable for this purpose.

The present invention relates to a method for determining analytes and to a device suitable for this purpose.

Immunoassays are an important part of clinically significant analysis methods, which are based on forming immunocomplexes from antigens and antibodies and are used for detecting analytes in liquid samples, for example in the field of medical, environmental, food and agricultural analytics. Because of the biophysical and biochemical properties of antigen-antibody binding, immunoassays generally provide high specificity and sensitivity with relatively simple equipment and at low costs, and thus have significant advantages over alternative detection methods.

A further field of application of immunoassays which is of significance in practice is detecting drugs in body fluids or on surfaces contaminated with drugs. In tests of this type, a sample of the analyte is usually taken from a surface, using a suitable sampling element, wherein the sampling element wetted with the analyte is brought into contact with a test strip, and the analyte released from the sampling element and transferred onto the test strip is detected using an immunological detection reaction.

One aspect which is of predominant importance especially in detecting drugs (for example amphetamines, metamphetamines, cannabis, cocaine, heroin) is the specificity, sensitivity and speed of the tests used. On the one hand, there is the need for highly sensitive detection methods so as to enable the assessement of the presence of drugs reliably and rapidly even for small sample volumes. On the other hand, the test formats should also have high specificity for the particular substance to be detected, so as to preclude false-positive measurement results and thus give an authoritative prediction about the specific drug in the tested substance it deals with.

EP 0 699 906 A2 discloses a rapid drug test, which is commercially available as a surface, sweat or saliva test, with the name DrugWipe® (from Securetec Detektionssysteme AG). The test uses a wiping element (“wiper”) essentially consisted of plastic with a welded fleece, by means of which a sample of the analyte is taken (for example from a surface or from a solution) and subsequently transferred (“lateral flow” technology) directly onto an immunochromatographic test strip which is stored in a disposable housing.

The chromatopgraphy is started by dipping the test strip in an aqueous solution (water or buffer comprising various reagents), and the result of the assessment can be read visually or using a suitable measurement device. The detection of the analyte is performed by the antibodies bound to drug molecules (antigens) binding on a test line and, because of its gold marking, forming a coloured line which can be detected visually in the readout window. Antibodies which do not carry drug molecules are captured by haptens present on the test strip in immobilised form, before reaching the test line.

By comparison with all the other rapid drug tests found on the market, DrugWipe® is the only product which forms a coloured line in the presence of a particular analyte in a sample, and thus presents a positive indication. All other commercial products show a negative indication, in which the non-appearance of a line is considered as positive detection of the respective analyte. A further advantage of DrugWipe® is the low sample volume required for detecting drugs and other substances. Whereas a sample volume of approximately 1-50 μl is sufficient for DrugWipe®, other commercial tests require a sample volume of at least 100 μl and up to several millilitres.

The low sample volume required for carrying out the DrugWipe® rapid drug test represents a decisive advantage over other commercially available test systems, since drug users often have a very dry mouth as a result of the physiological effect of the drugs, and can only actively bring forth a small amount of saliva. Thus, with drug users usually only very small sample volumes are available, which, apart from in the DrugWipe® rapid drug test, are generally not sufficient to obtain a reliable or an error-free result. In the cases it is at all possible to carry out a rapid drug test, the sampling often takes several minutes, which is finally not reasonable for the test subject.

The rapid test OraLab® 6, developed by Varian, is used to detect drugs from saliva samples, and is likewise based on lateral flow technology. Whilst the specificity of the test is about 90-100%, the sensitivity is only between 50 and 90% for amphetamines and opiates, and is sometimes even well below 50% for Δ⁹-tetrahydrocannabinol and cocaine (see DRUID study, published at TIAFT 2009 in Geneva). A further major drawback of this test is that relatively large sample volumes are required and are often not available with acute drug users.

According to the data of the DRUID study (TIAFT 2009, Geneva), the rapid test DrugCheck® 5000 from Dräger, which is also based on lateral flow technology, is a reliable test system for detecting drugs. However, this test format has the significant drawback that the results on the test strip can only be evaluated using a readout device, which is very expensive to acquire and is only in part suitable for rough use in the field. This test thus has major problems as regards cost-efficiency and simplicity of handling.

The test kit Rapid STAT, marketed by Mavand, is a further commercially available rapid drug test, in which a saliva sample diluted with buffer is incubated on an immunochromatographic test strip comprising marked binding partners and which according to the manufacturer's indications makes detection possible down to a lower limit of 15 ng/ml of Δ⁹-tetrahydrocannabinol.

A significant drawback of this test is the extremely complex and awkward handling thereof, which makes it unsuitable specifically for use by the traffic police, as well as the comparatively low specificity of 80-90% for N-tetrahydrocannabinol (see DRUID study, TIAFT 2009, Geneva). According to a study by the University of Mainz, the rate of false-positive test results for Δ⁹-tetrahydrocannabinol is more than 10% and the overall specificity is 84% (published at GTFCH 2009, Mosbach, http://www.gtfch.org/cms/images/stories/media/tk/tk76_{—}2/abstractsposter.pdf)

US 2005/0084855 A1 discloses a method for determining an analyte in a sample, which comprises bringing the sample into contact with a carrier capable of binding the analyte, bringing the analyte-containing carrier into contact with a stamp which contains a probe suitable for binding the analyte, and checking the amount of sample bound to the carrier on the probe. A major drawback of the detection method is that the stamp is not suitable for receiving a sample of the analyte from a surface to be analysed, and moreover, technical aids (such as a fluorescence scanner) are required for evaluating the results.

WO 2005/075982 A2 and US 2005/0175992 A1 each disclose a method for determining an analyte selected from pathogens and/or allergy-associated substances in a body fluid. Within the scope of the method, the body fluid is received using a wiping element, which is separate or integrated into a test element and which is brought into contact with the test element for a period of preferably 0.1 to 10 seconds, allowing at least part of the sample to be transferred from the wiping element onto the test element, and the analyte after the start of chromatography and the binding to an analyte-specific binding partner to be analysed. Further, a kit for detecting adenoviruses is disclosed, which comprises a chromatographic test strip in combination with a wiping element.

A decisive drawback of the detection method disclosed in WO 2005/075982 A2 and US 2005/0175992 A1 is that before the start of the chromatography no direct contact is produced between the analyte and the analyte-specific binding partner, meaning that the formation of a complex of analyte and analyte-specific binding partner can only be completed during the progression of the chromatography. Moreover, before the binding to the analyte-specific binding partner, which takes place in the test element, the body fluid containing the analyte is not prepared in any way, for example by chemical and/or mechanical pre-treatment of the sample, meaning that in practice optimal binding between the analyte and the analyte-specific binding partner cannot be achieved.

However, biological samples with a complex chemical composition (for example blood, saliva or sweat) are particularly subject to considerable fluctuation among individuals, for example in consistency, viscosity, salt content and/or protein content, and offer a variety of non-specific binding sites for the analyte. If a biological sample is not pre-treated before the start of the chromatography, this leads to poorly reproducible results and to drawbacks as regards the sensitivity of the method, since the analyte is no longer available for actual analyte detection, as a result of the binding to non-specific binding sites in the sample matrix.

EP 0 811 842 A2 and EP 0 699 906 A2 disclose a method for detecting an analyte in a body fluid or on a contaminated surface. To carry out the method, in each case a test kit is used which comprises a test strip of one or more capillary-active, chromatography-capable materials, a sampling element separate from the test strip surface, as well as a pressing device for bringing the test strip surface and sampling element into contact, wherein the sampling element is moistened with an aqueous buffer, a detergent and/or an organic solvent, for better receiving the analyte.

EP 2 381 258 A1 discloses a method for determining an analyte, which comprises providing a microfluidic analysis device, taking a sample containing the analyte from the sample surface, using a wiping element, introducing the analyte-containing wiping element into the microfluidic analysis device, eluting the analyte from the wiping element within the microfluidic analysis device using an eluent, and determining the analyte on a test element stored in the microfluidic analysis device. In a preferred variant, the wiping element comprises a transfer reagent which contains at least one protein, at least one carbohydrate, at least one sugar alcohol and/or at least one salt.

WO 2005/121793 A2 discloses a method for detecting a metamphetamine in a liquid sample. To carry out the method, a kit is preferably used, which comprises a chromatographic test strip and optionally a sampling element. The test strip for its part comprises (a) a dry, porous material, on which a pseudoephedrine/carrier conjugate or an antibody, which can bind to both the metamphetamine and the conjugate, is immobilised in a detection zone, and (b) a marker release zone separated therefrom, which can release either the antibody in a marked form if the detection zone contains immobilised pseudoephedrine/carrier conjugate, or detectable pseudoephedrine/carrier conjugate if the detection zone contains an immobilised antibody, into the liquid.

The detection methods disclosed in EP 0 699 906 A2, EP 0 811 842 A2, EP 2 381 258 A1 and WO 2005/121793 A2 also have the drawback that no pre-treatment of the sample is carried out before the start of chromatography and/or no direct or defined incubation between the analyte and the analyte-specific binding partner takes place. Instead, the analyte and the analyte-specific binding partner only come into contact with one another during the chromatography, meaning that optimal binding between the analyte and the analyte-specific binding partner cannot be achieved, and that there is ultimately a negative effect on the sensitivity and specificity of the method, in particular when detecting poorly water-soluble analytes. This means at least in some cases that the legally required minimum detection thresholds for drug molecules are not met.

Starting from the above-disclosed test systems, the problem underlying the present invention was therefore to provide a method for determining an analyte, in particular for determining drugs, in which the drawbacks of the prior art are overcome at least in part. In particular, the method should have high sensitivity and specificity for all analytes to be tested, have easy handling, and make rapid determination of the analytes possible without the use of additional technical aids.

This object is achieved according to the invention by a method for determining an analyte, comprising the steps of:

(a) providing a test element, comprising
(i) a first region provided for receiving the eluent,
(ii) a second region provided for applying a sample containing the analyte, and which comprises a binding partner specific to the analyte,
(iii) a third region provided for the visual detection of the analyte,
(iv) a fourth region provided for receiving the excess of eluent, and
(v) optionally a housing,

(b) receiving a sample containing the analyte from a sample surface, using a wiping element,

(c) bringing the wiping element into contact with the second region of the test element for a period of at least 10 seconds, wherein part of the sample is transferred from the wiping element to the test element and the analyte is incubated with the analyte-specific binding partner,

(d) bringing the first region of the incubated test element into contact with an eluent, and

(e) determining the presence and/or the amount of the analyte, wherein the test element and/or the wiping element comprise an analyte transfer reagent which contains at least one analyte-non-specific substance selected from the group consisting of a protein, a protein mixture, a carbohydrate and a sugar alcohol.

Surprisingly, it has been found in the context of the present invention that, by the method according to the invention, analytes can be detected in a simple and reproducible manner with high sensitivity and specificity, without large sample amounts of the analyte and complex technical aids, for example for reading the measurement results, being required.

The first step of the method according to the invention requires the preparation of a test element which is suitable for determining the analyte and which comprises a binding partner specific to the analyte. The test elements used for this purpose are in principle of any physical form familiar to the person skilled in the art, which is suitable for determining the presence and/or the amount of an analyte in a sample. In this context, the test element is preferably designed in such a way that, in the presence of the analyte to be determined, it produces a visually detectable signal which makes qualitative and/or quantitative determination of the analyte possible. Examples of test elements within the meaning of the present invention include, among others, test strips, test bands and test pads, to which the analyte may for example be applied in the form of an aqueous or non-aqueous solution.

In a preferred embodiment, the test element is a chromatographic test strip which, in a variant of the invention, may be formed of a single chromatography-compatible, optionally strip-shaped material. Preferably, however, the chromatographic test strip comprises a plurality of capillary-active faces, arranged overlapping on a carrier layer, of the same or different chromatographic materials, which are in fluid communication with one another and thus form a transport path along which a fluid, driven by capillary forces, can flow through all of the regions of the test element. Any known liquid-absorbing, porous or capillary-active material may be used as the chromatography material, such as cellulose and derivatives thereof, glass fibres, as well as fleece and fabrics of synthetic or natural materials. Chromatographic test strips which may be used in the context of the present invention are disclosed for example in EP 0 699 906 A2, to the disclosure of which reference is hereby explicitly made.

The analyte-specific binding partner may be any chemical substance which binds to the analyte to be tested but does not form any binding to other substances which may be present in the sample and/or in the test element. Chemical substances which satisfy these requirements are generally known to the person skilled in the art or may be produced by routine experiments and using known techniques in accordance with the requirements of the respective test element. Preferably, the analyte-specific binding partner is capable of migration in the test elements disclosed herein, and can for example be immobilised on the test element by means of the suitable positioning of a capture reagent at a predetermined position on the test element. Analyte-specific binding partners, the use of which has been found to be particularly advantageous, are disclosed for example in EP 1 579 222 B1, to the disclosure of which reference is hereby explicitly made.

In a preferred embodiment, the analyte-specific binding partner is an antibody or a functional antibody fragment, which may optionally additionally have a binding site or a plurality of binding sites for a capture reagent as disclosed above. The term “functional antibody fragment” as used in the context of the present application denotes an antibody fragment which can perform the intended function thereof of specific binding to the analyte. By contrast, a “non-functional antibody fragment” denotes an antibody fragment which does not form binding or specific binding to the analyte and thus does not perform the intended function of specific binding to the analyte.

To make it easier to detect the analyte, using the test elements disclosed herein, the analyte-specific binding partner may optionally comprise a detectable marking, such as an enzyme marking, dye marking, fluorescence marking, metal marking or particle marking. For the purposes of the present invention, the use of analyte-specific binding partners which comprise a visually detectable marking, in particular a metal marking, has been found to be advantageous. The marking is more preferably a gold marking, which has the advantage that the test result can be visually detected and evaluated directly by the user. Techniques by which the above-disclosed markings can be introduced into a molecule to be marked are known to the person skilled in the art and are therefore not described in greater detail.

To determine the analyte, using the test elements disclosed herein, in a further step of the method according to the invention a sample containing the analyte is received from a surface to be analysed (for example tongue, skin, other surface), using a suitable wiping element. Any element which can receive a sample of the analyte and makes subsequent transfer of the sample to the test element possible, for example using capillary effects, may be used as the wiping element. The wiping element comprises one or a plurality of (mutually independent) wiping faces, 2, 3 or 4 wiping faces being preferred if there is a plurality of wiping faces. If a wiping element having a plurality of wiping faces is used, one test element preferably comes into contact with each wiping face of the wiping element when the test device comprising a plurality of (mutually independent) test elements is subsequently brought together with the wiping element.

The at least one wiping face, which is preferably fused to a surface of the wiping element, may in principle consist of any material which appears expedient to the person skilled in the art for the purposes of the present invention and makes it possible both to administer the analyte to the wiping face and subsequently to transfer the analyte to the test element. Especially absorbent materials, in particular fabrics, fleece and/or porous matrices (for example membranes and sponges) have proved here to be expedient. In the context of the invention, fleece are more preferably used, in particular fleece based on cellulose, polyester and/or glass fibres, wherein the fibres are optionally being held together using an organic binder. Suitable fleece and fibres are disclosed for example in DE 38 02 366 A1 and EP 0 699 906 A2, to the disclosure of which reference is hereby explicitly made.

As regards the external configuration of the wiping face(s), there are in principle no limitations on thickness, size and shape. The thickness of the wiping face(s) or of the material used therefore is respectively of lesser importance for the purposes of the present invention, and is usually in a range of 0.1 to 3 mm. The size of the wiping face(s) is advantageously adapted to the size of the test element, in other words the width of the wiping face(s) should neither exceed nor fall below the width of the test element. Preferred sizes of the wiping faces are in the range of 0.3 to 2 cm for the length and in the range of 0.3 to 1 cm for the width. The shape of the wiping face(s) can be adapted to the respective requirements of the surface to be analysed, wherein a triangular, quadrilateral (for example square, rectangular, diamond-shaped) or roller-shaped configuration of the wiping face(s) is considered particularly advantageous. A roller-shaped configuration of the wiping face(s) has the advantage that due to the large surface of the wiping face(s), a particularly good contact is formed between the wiping element and the second region of the test element, and accordingly an increase in the sensitivity of the method can be achieved.

So as to ensure particularly high sensitivity and specificity in determining the analyte, the method according to the invention provides that the test element and/or the wiping element comprises an analyte transfer reagent. In one embodiment of the invention the test element used for determining the analyte comprises the analyte transfer reagent, whilst in another embodiment the wiping element contains the analyte transfer reagent. More preferably, however, both the test element and the wiping element comprise an analyte transfer reagent as defined in detail in the following.

The analyte transfer reagent contains at least one analyte-non-specific substance selected from the group consisting of a protein, a protein mixture, a carbohydrate and a sugar alcohol, the term “analyte-non-specific substance” denoting a chemical substance which does not exclusively bind to the analyte to be tested, but can also form bonds to other substances which may be present in the sample and/or in the test element. The concentration of protein, protein mixture, carbohydrate or sugar alcohol in the analyte transfer reagent(s) can be adapted respectively by the person skilled in the art in accordance with the respective requirements of the test element, but is usually approximately 0.01 to approximately 15 wt. % based on the total weight of the analyte transfer reagent.

If the test element comprises the analyte transfer reagent, it is further considered preferable for the analyte transfer reagent to contain a carbohydrate and/or a sugar alcohol, in particular a sugar alcohol. By contrast, the wiping element preferably comprises an analyte transfer reagent which contains an analyte-non-specific protein and/or an analyte-non-specific protein mixture, in particular an analyte-non-specific protein. In the test elements and/or wiping elements disclosed herein, an albumin, in particular ovalbumin or bovine serum albumin, is preferably used as the analyte-non-specific protein, whilst for example gelatines or skimmed milk powder may be used as the analyte-non-specific protein mixture.

The term “carbohydrate” as used in the present application denotes monosaccharides and oligosaccharides, in particular disaccharides and trisaccharides, of the general molecular formula CₙH₂ₙOₙ, which may in each case be of natural or synthetic origin. In particular naturally occurring tetroses, pentoses and hexoses are used as monosaccharides, such as erythrose, threose, ribose, arabinose, lyxose, xylose, allose, altrose, galactose, glucose, gulose, idose, mannose, talose and fructose, which may each be present in the D or the L form. In particular naturally occurring disaccharides and trisaccharides may be used as oligosaccharides, such as lactose, maltose, saccharose, trehalose, gentianose, kestose and raffinose. In a particularly preferred embodiment of the invention, the carbohydrate is a substance selected from the group consisting of glucose, lactose, maltose, mannose and saccharose.

The term “sugar alcohol” as used in the present application denotes monosaccharide sugar alcohols of the general molecular formula CₙH₂ₙ₊₂Oₙ and disaccharide alcohols of the general molecular formula CₙH₂ₙOₙ₋₁, which may in each case be of natural or synthetic origin. Preferred monosaccharide sugar alcohols comprise glycerol, erythritol, threitol, ribitol, arabinitol, xylitol, allitol, altritol, galactitol, glucitol, iditol and mannitol, which may each be present in the D or the L form. In particular isomalt, lactitol and maltitol may be used as disaccharide sugar alcohols. In a particularly preferred embodiment of the invention, the sugar alcohol is a substance selected from the group consisting of glucitol, glycerol, lacitol, mannitol and xylitol.

The analyte transfer reagent, which promotes the transfer of the analyte from the surface to be analysed to the wiping element and/or the subsequent transfer of the analyte from the wiping element to the test element, in particular by blocking free binding sites on the wiping element and/or by influencing the analyte properties, may for example be impregnated on the test element and/or the wiping element, in particular in the region of the wiping face(s). Techniques which may be used for applying the analyte transfer reagent to the test element and/or the wiping element are known to the person skilled in the art, and may be selected in a targeted manner in accordance with the requirements of the test element.

So as to ensure that the analyte is received as completely as possible, the at least one wiping face of the wiping element may be moistened with a small amount of a suitable liquid, for example approximately 1-100 μl, when dry surfaces are being sampled. For this purpose, the liquid may for example be applied to the wiping face(s), using a pipette, an automatic metering device or a material which releases the liquid (for example a sponge impregnated with the liquid). Alternatively, the wiping face(s) may also be moistened in such a way that the at least one wiping face comprises the liquid in a microcapsulated or blister-packed form and the liquid is released by suitable measures, for example by pressing the wiping face(s) onto the surface to be analysed. Liquids which are suitable for moistening the wiping face(s) comprise in particular water and aqueous buffer solutions, which may optionally contain further substances, such as a detergent and/or an organic solvent, as disclosed above respectively. If the wiping element is brought into contact with moist surfaces or liquid samples, prior moistening of the wiping face(s) is generally not required.

Once the sample has been taken, in a further step of the method according to the invention the wiping element is brought into contact with the second region of the test element comprising the analyte-specific binding partner, preferably by light mechanical pressing of its wiping face(s), wherein at least part of the sample is transferred from the wiping element to the test element. The pressure with which the wiping element is pressed onto the test element should be at least great enough for there to be planar contact between the surface of the test element and the wiping face(s) of the wiping element and thus for fluid communication between the two elements to be made possible. As a result of the direct contact between the wiping element and the second region of the test element, a direct and thus more rapid contact between the analyte and the analyte-specific binding partner occurs, allowing a rapid formation of the complex of the analyte and the analyte-specific binding partner to occur and whereby the sensitivity and specificity of the detection method are significantly improved.

So as further to improve the sensitivity and specificity of the method, in a preferred variant the second region of the test element, designed for applying the sample, additionally comprises at least one means which produces a chemical and/or mechanical treatment of the sample containing the analyte, for example by blocking or destroying non-specific binding sites in the sample and/or by altering the sample consistency. The analyte is thus optimally available for binding to the analyte-specific binding partner (for example to an antibody), improving the accessibility of the analyte for the analyte-specific binding partner, as well as transport across the individual regions of the test element. When a chemical sample treatment means is used, this can for example be impregnated on the test element, wherein an aqueous or non-aqueous solution, which contains the chemical sample treatment means in a concentration of approximately 0.01 to approximately 5 wt. %, is preferably used for the impregnation. More preferably, at least one chemical sample treatment means and at least one mechanical sample treatment means are used in parallel in the test element.

Chemical sample treatment means which may be used in the context of the method according to the invention comprise in particular acids, bases, buffers, organic solvents and detergents, wherein bases and detergents are considered particularly preferred. Specific examples of acids comprise inorganic acids (for example hydrochloric acid) and organic acids (for example acetic acid and citric acid). Examples of bases comprise in particular alkali metal and alkaline earth metal hydroxides, such as sodium hydroxide and calcium hydroxide, whilst calcium carbonate, Tris, PBS, phosphate buffer, borate buffer, BICINE buffer and HEPES, among others, may be used as buffer. Examples of detergents comprise, among others, octyl glucoside, cholamidopropane sulphonate, polidocanol, polyalkylene glycol ether (for example Brij®, Synperonic®) and polysorbates (for example Tween® 20, Tween® 80). Examples of organic solvents comprise in particular dimethylsulphoxide, ethanol, glycerine, isopropanol, methanol and mixtures thereof.

Mechanical sample treatment means which may be used in the context of the method according to the invention comprise for example fabrics and/or fleece, in particular fleece which filter or separate out the sample before the analyte is incubated with the analyte-specific binding partner, in such a way that in particular solid and viscous sample constituents (for example solid and viscous saliva components), which may negatively influence the detection method, are held back. Specific examples of fleece which can produce a mechanical treatment of the sample comprise for example the commercially available products Ahlstrom 8964, Whatman Rapid 24Q and Freudenberg FS 2216, but are not limited thereto.

To make it easier for the user to bring the test element and wiping element into contact, in one embodiment of the invention one or a plurality of test elements may be placed in a housing. The housing preferably has at least one opening, via which the wiping element can be brought into contact with the region of the respective test element provided for applying the sample. If the wiping element has a plurality of wiping faces, it will be preferred that the housing merely contains a single opening for receiving the wiping element. Alternatively, however, it is also possible that the housing contains a number of openings corresponding to the number of wiping faces.

The opening(s) may be arranged in any desired shape (with respect to one another), and be of any size and shape appearing suitable to the person skilled in the art, but are usually adapted to the arrangement, size and shape of the wiping face(s) of the wiping element. As a result, it is possible for example to bring the wiping element and test element together in a predefined manner, so as to prevent inappropriate use of the test system (see FIG. 3).

In a further embodiment, the housing may further comprise a holding device, which makes reversible fixing of the wiping element on the housing possible, in such a way that said wiping element can for example be removed for sampling, and brought into the housing again after sampling. In this context, by selecting a suitable position for the holding device, the wiping element can be placed on the housing in such a way that intensive contact between the wiping element, or its wiping face(s) respectively, and the test element is ensured, providing good transfer of the analyte from the wiping element to the test element comprising the analyte-specific binding partner.

According to the invention, the test element and the wiping element wetted with the analyte are brought into contact for a period of at least 10 seconds for the purpose of the high sensitivity and specificity of the analyte determination, wherein the analyte-specific binding partner located on the test element is incubated with the analyte to be determined, and optionally in addition a chemical or mechanical treatment of the sample containing the analyte occurs. This ensures that the analyte on the one hand is released as completely as possible from the sample matrix of the wiping element and on the other hand can react almost quantitatively with the analyte-specific binding partner. In this connection, an incubation time of approximately 10 seconds to approximately 600 seconds, more preferably approximately 30 seconds to approximately 300 seconds, most preferably approximately 60 seconds to approximately 180 seconds, has been found to be advantageous. As a result of carrying out the method in this manner, the sample volume required for determining the analyte can usually be reduced to less than 10 μl, and the sensitivity of the test system can be greatly improved.

After incubation with the analyte, the test element is brought into contact with an eluent. For this purpose, the test element preferably comprises an end region which is provided for receiving the eluent and usually comprises an absorbent material, such as fabric and/or fleece. After this region is wetted with the eluent, the eluent migrates through the various regions of the test element, wherein the analyte, the analyte-specific binding partner as well as complexes thereof are being transported accordingly. The capillary action of the individual components of the test element, which are arranged or interconnected respectively in such a way that an uninterrupted flow of eluent is provided, may here advantageously be expoited.

In the context of the present invention, any eluent appearing suitable to the person skilled in the art may be basically used as the eluent. Preferably, water and aqueous buffer solutions, which may optionally comprise other substances, such as a carbohydrate, a sugar alcohol, a detergent and/or an organic solvent, as respectively disclosed above, at a concentration of usually approximately 0.05 to approximately 1.5 wt. %, are, however, used in the method disclosed herein. In a preferred embodiment of the invention, the eluent comprises a sugar alcohol, as defined above, and/or a derivative thereof, in which at least one hydroxyl group of the sugar alcohol is exchanged for a mercapto group. In the context of the present invention, a preferably aqueous eluent which comprises 1,4-dithioerythritol as a constituent is considered particularly preferred, and an increase in the sensitivity and/or specificity of the analyte determination can be achieved through its use.

When the test element is introduced into a housing, there are various options for wetting the test element with eluent depending on the configuration of the housing. For example, if the test element is completely mounted in the housing, the eluent can for example be applied to the first region of the test element, which is formed for receiving eluent, by pressing on an ampoule containing the eluent, which is preferably stored inside the housing. However, if the first region of the test element, which is provided for receiving eluent, stands out of the housing, there is the option of dipping the region into the eluent.

After the test element is brought into contact with the eluent, the determination of the analyte is initiated, which preferably takes places immunologically and comprises for example a competitive test format and/or a non-competitive test format (sandwich test format), in particular a combination of a competitive test format and a non-competitive test format. The detection method according to the invention usually initially comprises forming a complex of analyte molecules and an analyte-specific, optionally marked binding partner, which is transported onwards by means of the eluent, for example together with an unbound binding partner and/or further substances present in the sample, to a region of the test element which is provided for the visual detection of the analyte.

The region provided for the visual detection of the analyte usually comprises a plurality of defined portions in which different reagents can be immobilised.

In one embodiment, this region comprises a portion which is provided for the binding of the unbound analyte-specific binding partner, one portion which is provided for the binding of the complex of analyte and analyte-specific binding partner, and optionally a portion in which a control signal is produced independently from the analyte (see FIG. 1). The region provided for the visual detection of the analyte may be formed from one or a plurality of materials which appear suitable to the person skilled in the art for the purposes of the invention, such as, for example, Nylon®, nitrocellulose or polyvinylidene fluoride membranes.

The portion provided for the binding of the unbound analyte-specific binding partner may for example comprise immobilised analyte analogues, in particular polyhaptens, which capture the analyte-specific, optionally marked binding partner as a result of the formation of a complex at a defined position on the test element (capture line), and thus prevent the production of false-positive results. The complex of analyte and analyte-specific binding partner is usually not immobilised on the capture line, since the analyte blocks the binding sites required for this purpose on the analyte-specific binding partner.

The portion provided for the binding of the complex of analyte and analyte-specific binding partner preferably comprises an optionally marked binding partner which is specific to the analyte-specific binding partner, and which leads to the immobilisation of the complex of analyte and analyte-specific binding partner at a predetermined position on the test element (test line) and thus makes the visual determination of the analyte possible. The complex is generally immobilised using a free binding site of the analyte-specific binding partner, wherein the positive detection of the analyte results in the colouring of the test line.

So as to be able to read the signal on the test line unambiguously, and prevent confusion with the capture line, the capture line may optionally be covered in a suitable manner. If the test element further comprises a control portion, a control line, which acts as an indicator that the test element is functioning without faults, additionally appears when the method is carried out. Excess eluent, which leaves the region of the test element formed for the visual detection of the analyte, can be received in a region of the test element provided specifically for this purpose, by way of a liquid-absorbing material.

In another embodiment, the region provided for the visual detection of the analyte, as disclosed above, does not comprise a portion provided for the binding of unbound analyte-specific binding partner (see FIG. 2). In this case, a special analyte-specific binding partner, as disclosed for example in EP 1 579 222 B1, is preferably used to produce the complex of analyte and analyte-specific binding partner. Complexes of this type can therefore be immobilised, using a complex-specific binding partner, and this ultimately leads to a simplified detection of the analyte, since the production of false-positive signals, which can be caused among other things by an analyte-specific binding partner which is not bound at the capture line, is prevented.

In this case, the portion provided for the binding of the complex of analyte and analyte-specific binding partner preferably comprises a complex-specific binding partner, which leads to the immobilisation of the complex of analyte and analyte-specific binding partner at a predetermined position on the test element (test line) and thus makes a visual determination of the analyte possible. The immobilisation of the complex of analyte and analyte-specific binding partner generally takes place by way of a free binding site of the complex-specific binding partner, wherein the colouring of the test line is accompanied by the positive detection of the analyte.

The qualitative and/or quantitative determination of the analyte, carried out in the final step of the method according to the invention, may take place in any user-defined way. In principle, any methods known from the prior art for detecting complex formation, which produce a measurable signal which can be evaluated or read manually or using suitable means respectively, may be used for this purpose. In the context of the present invention, visual detection methods are preferably used, in particular photometric or fluorimetric detection methods. According to the invention, visual detection of the analyte is particularly preferred.

In a particularly preferred embodiment, a plurality of different analytes, in particular 2 to 20 different analytes, are determined simultaneously by the method according to the invention. In this case, in the second region provided for applying the sample, the test element usually comprises a number of different analyte-specific binding partners, corresponding to the number of different analytes, and, if the test element does not contain a portion for capturing unbound analyte-specific binding partners, optionally a number of complex-specific binding partners, as disclosed above, corresponding to the number of different analytes. It can thus be ensured that the parallel determination of the different analytes on the test element is performed essentially independently from each other and that no interference occurs.

The method according to the invention makes it possible to determine one or more analytes with high sensitivity and specificity. It is thus preferable according to the invention to determine analytes with a specificity of at least 95% and/or a sensitivity of at least 90%. More preferably, the determination takes place with a specificity of at least 98% and/or a sensitivity of at least 95%, in such a way that analytes can be detected down to a lower detection threshold of approximately 1 ng/ml sample, using the method disclosed herein.

According to the invention, it is further preferred for the test element and/or wiping element used in the method disclosed herein to be sterile before contact with the sample containing the analyte. In this context, the test element and/or the wiping element may be inserted into a suitable storage container before or after sterilisation, wherein a corresponding insertion prior to carrying out sterilisation is considered preferred. The sterilisation itself can take place in various ways, and comprises for example chemical sterilisation, heat sterilisation, and sterilisation by ionising radiation. The sterilised test element, optionally together with the wiping element, is preferably packed in a sterile manner after sterilisation. The sterile packing makes it possible to keep both the test element and the wiping element sterile until subsequent use, without the need for repeated sterilisation. More preferably, the test element and wiping element disclosed herein are therefore disposable articles, which are not reused after use.

The method according to the invention can be used for determining any biological or chemical substance which is detectable, for example using immunological techniques. Preferably, however, the method disclosed herein is used for detecting narcotics as listed in the German law on narcotics. Specific examples of such narcotics comprise, among others, dissociatives, deliriants, empathogens, entactogens, hypnotics, narcotics, psychedelics, sedatives and stimulants, but are not limited thereto. In a particularly preferred embodiment according to the invention at least one analyte selected from the group consisting of amphetamines, benzodiazepines, cannabinoids, in particular Δ⁹-tetrahydrocannabinol, ketamines, metamphetamines, opiates, in particular morphine, codeine or dihydrocodeine, natural or synthetic opioids, in particular heroin, and tropane alkaloids, in particular cocaine, are determined, wherein Δ⁹-tetrahydrocannabinol and cocaine are particularly preferred as analytes.

The analyte may be from any desired source, such as a surface of an item wetted with the analyte or a body fluid, such as whole blood, plasma, serum, urine, saliva or sweat. The presence and/or amount of an analyte is/are preferably determined from a sample of whole blood, urine or saliva using the method disclosed herein. The sample amount required for carrying out the method is usually approximately 0.01 μl to approximately 100 μl, preferably approximately 0.05 μl to approximately 20 μl, more preferably approximately 0.1 μl to ≦10 μl, and most preferably approximately 0.5 μl to approximately 5 μl.

A further aspect of the invention relates to a test element for determining an analyte, comprising:

(i) a first region provided for receiving the eluent,

(ii) a second region provided for applying a sample containing the analyte, and which comprises a binding partner specific to the analyte,

(iii) a third region provided for the visual detection of the analyte,

(iv) a fourth region provided for receiving the excess of eluent,

(v) optionally a housing, and

(vi) an analyte transfer reagent which contains at least one analyte-non-specific substance selected from the group consisting of a protein, a protein mixture, a carbohydrate and a sugar alcohol.

Another further aspect of the invention relates to a wiping element for receiving an analyte from a surface, comprising an analyte transfer reagent which contains at least one analyte-non-specific substance selected from the group consisting of a protein, a protein mixture, a carbohydrate and a sugar alcohol.

Another further aspect of the invention relates to a kit for determining an analyte, which is preferably used for carrying out the above-disclosed method and comprises the following components:

(a) a test element, comprising
(i) a first region which provided for receiving the eluent,
(ii) a second region provided for applying a sample containing the analyte, and which comprises a binding partner specific to the analyte,
(iii) a third region provided for the visual detection of the analyte,
(iv) a fourth region provided for receiving the excess of eluent, and
(v) optionally a housing, and

(b) a wiping element provided for receiving a sample of the analyte, the test element and/or the wiping element comprising an analyte transfer reagent which contains at least one analyte-non-specific substance selected from the group consisting of a protein, a protein, mixture, a carbohydrate and a sugar alcohol.

As regards preferred configurations of the test element according to the invention, the wiping element according to the invention and the test element and wiping element contained in the kit according to the invention respectively, reference is made to the statements made in connection with the description of the method according to the invention.

The invention is to be described in greater detail by way of the following drawings and examples.

### DESCRIPTION OF THE DRAWINGS

FIG. 1 is a cross section through an embodiment of a test element for carrying out the method according to the present invention. The test element, shown in the form of a test strip, comprises:

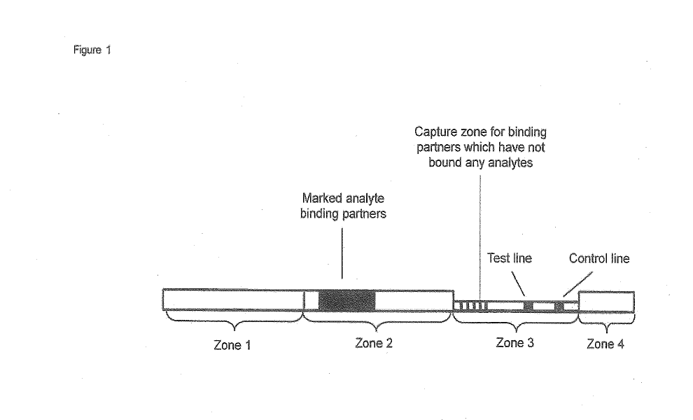

(a) a first region provided for receiving the eluent (zone 1),

(b) a second region provided for applying a sample containing the analyte, and which comprises an analyte-specific, marked binding partner as well as optionally further reagents (zone 2),

(c) a third region provided for the visual detection of the analyte and which comprises a capture line, a test line and a control line (zone 3), and

(d) a fourth region provided for receiving the excess of eluent (zone 4).

FIG. 2 is a cross section through a further embodiment of a test element for carrying out the method according to the present invention. The test element, shown in the form of a test strip, comprises:

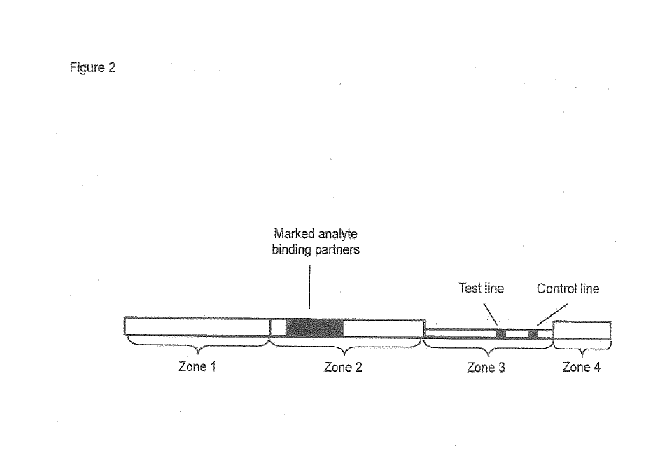

(a) a first region provided for receiving the eluent (zone 1),

(b) a second region provided for applying a sample containing the analyte, and which comprises an analyte-specific, marked binding partner and optionally further reagents (zone 2),

(c) a third region provided the visual detection of the analyte and which comprises a test line as well as a control line (zone 3), and

(d) a fourth region provided for receiving the excess of eluent (zone 4).

FIG. 3 is an illustration of an embodiment of a wiping element and a test device for carrying out the method according to the present invention. The wiping element comprises three wiping faces (each of a triangular shape) spatially separated from each other for receiving at least 3 different analytes. The test device comprises three test elements (each in the form of a test strip) spatially separated from each other as well as a housing which contains the test elements, which for its part comprises an opening for reading the test results and as well as an opening for bringing the wiping element into contact with the test elements.

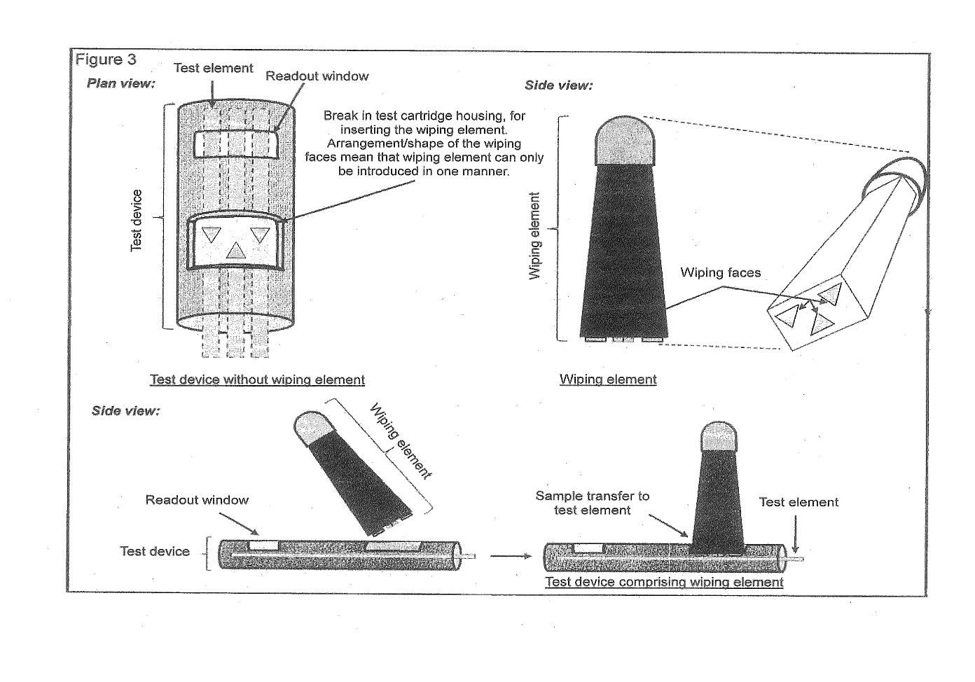

FIG. 4 is an illustration of a further embodiment of a wiping element and a test device for carrying out the method according to the present invention. The wiping element comprises three wiping faces (each of a roller shape) spatially separated from each other for receiving at least 3 different analytes. The test device comprises three test elements (each in the form of a test strip) spatially separated from each other and a housing which contains the test elements, which for its part comprises an opening for reading the test results as well as an opening for bringing the wiping element into contact with the test elements.

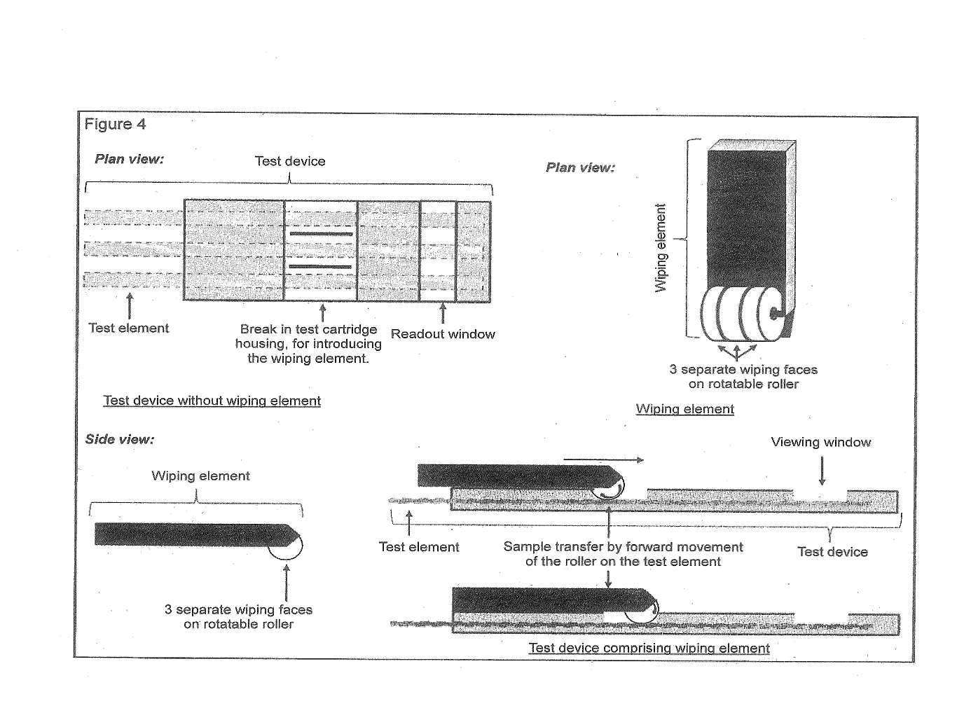

### EXAMPLES

### Example 1

### Producing Δ⁹-THC Immunogen

1.25 ml of a parent solution of g-tetrahydrocannabinolic acid A (1-hydroxy-(−)-(6aR,10aR)-6,6,9-trimethyl-3-pentyl-6a,7,8,10a-tetrahydrobenzo[c]chromene-2-carboxylic acid, from Lipomed) in N,N-dimethylformamide (concentration: 100.8 mg/ml) was diluted with 8.75 ml of N,N-dimethylformamide to a final volume of 10 ml (final concentration: 12.6 mg/ml).

5 ml of this diluted solution was subsequently added, under stirring, to an aqueous solution of bovine serum albumin (BSA), which had been prepared previously by dissolving 250 mg bovine serum albumin (from Sigma Aldrich) in a mixture of 25 ml distilled water and 2.5 ml N,N-dimethylformamide. After adding 6.3 ml of distilled water to the resulting reaction mixture, the remaining 5 ml of the above-described diluted solution of g-tetrahydrocannabinolic acid A in N,N-dimethylformamide, a further 6 ml of distilled water, and 0.2 ml of a solution of 1-ethyl-3-(3-dimethylaminopropyl)carbodiimide-hydrochloride (from Sigma Aldrich) in N,N-dimethylformamide (concentration 500 mg/ml) were consecutively added.

The beaker containing the solution was wrapped in aluminium film and stirred with a magnetic stirrer at 5° C. for 24 h. Subsequently, the solution was transferred into a dialysis container, and dialysed over a period of 4 days at 5° C. against a 25% solution of N,N-dimethylformamide in water. The dialysate was tested by immunoelectrophoresis. Incubation with a goat anti-BSA antibody (from Genetex) and a precipitate band showed that the g-m THC immunogen was distinguished from the band of the bovine serum albumin.

### Example 2

### Extraction of the Antibodies Against Δ⁹-THC

20 mice were immunised with the Δ⁹-THC immunogen obtained in Example 1 in complete Freund's adjuvant (from Sigma Aldrich), wherein the dose for the first and each subsequent immunisation was 75 μg immunogen per animal in each case. The immunisations were carried out over a period of 6 months, at an interval of one month in each case.

The sera obtained from the immunised mice were subsequently analysed in a microtitre plate assay for the presence of antibodies against Δ⁹-THC. For this purpose, microtitre plates coated with streptavidin were initially incubated at 4° C. for 12 h with 10 ml of a solution of Δ⁹-tetrahydrocannabinol-[N-biotinylaminocaproyl-(3,6-dioxa-8-aminooctyl)-amide] in phosphate-buffered saline solution, which had been produced previously by reacting Δ⁹-tetrahydrocannabinol succinimidyl ester and N-(biotinylaminocaproyl)-1,8-diamino-3,6-dioxaoctane (from Applichem).

After washing with 20 ml of a 0.05% solution of Tween 20 in phosphate-buffered saline solution, the microtitre plates were each incubated at 20° C. for 1 h with 10 ml of the sera to be analysed, and subsequently washed again with 20 ml of a 0.05% solution of Tween 20 in phosphate-buffered saline solution. The detection was carried out by incubating at 20° C. for 1 h with 10 ml of a solution of a conjugate of peroxidase and rabbit anti-mouse IgG (from Dako), washing it with 20 ml of a 0.05% solution of Tween 20 in phosphate-buffered saline solution, and displacing with the substrate. Sera having a good affinity towards Δ⁹-THC were selected for the extraction of antibodies against Δ⁹-THC.

### Example 3

### Production of a Gold-Marked Antibody Against Δ⁹-THC

Gold sol having a particle diameter of 20 nm as determined by photon correlation spectroscopy was prepared in accordance with methods of the state of the art (Frens, Nature (1973), 241, 20-22). The Δ⁹-THC-specific antibodies obtained in example 2 were marked with the gold particles in accordance with known methods (Geoghegan et al., J. Immunol. Meth. (1980), 34, 11-31).

### Example 4

### Use of Glucitol to Increase Sensitivity and Specificity

A region formed of fleece of a chromatographic test strip, which contained a Δ⁹-THC-specific antibody in accordance with example 3, was impregnated with three differently concentrated solutions of glucitol (from Sigma) in distilled water.

Subsequently, pooled saliva (saliva mix from five people) was set to a Δ⁹-THC concentration of 100 ng/ml using a solution of Δ⁹-THC (from Cerilliant) diluted in methanol. After applying 10 μl of this doped pooled saliva to the wiping face of a standard wiping element (from Securetec Detektionssysteme), the wiping element was brought into contact with the impregnated region of the chromatographic test strip, and the test strip was incubated at room temperature for a period of 60 seconds.

After incubation with the sample of the analyte, the test strip was dipped in tap water for 15 seconds, and the chromatography process was thus started.

The completion of the test was reached within 5 to 10 minutes. The results of the three determinations are shown in Table 1:

TABLE 1

| Glucitol concentration | |
|---|---|
| (wt. % in the | Test line signal |
| impregnated solution) | for Δ⁹-THC |
| 2% | Positive |
| 1% | Weak positive |
| 0% | Negative |

As can be seen from Table 1, impregnating the fleece with increasing concentrations of glucitol leads to improved saliva transfer from the wiping element to the chromatographic test strip. At the same time, glucitol improves the release of Δ⁹-THC from the saliva sample.

Together, these make improved contact possible between Δ⁹-THC and the marked antibody provided in the contacted region of the test strip, achieving much more specific and sensitive binding of Δ⁹-THC to the specific binding partner and leading to stronger signal development on the test line of the chromatographic test strip.

### Example 5

### Use of 1,4-Dithioerythritol in the Eluent for Increasing Sensitivity and Specificity

Pooled saliva (saliva mix from five people) was set to a Δ⁹-THC concentration of 80 ng/ml, using a solution of Δ⁹-THC (from Cerilliant) diluted in methanol. After applying 10 μl of this doped pooled saliva to the wiping face of a standard wiping element (from Securetec Detektionssysteme), the wiping element was brought into contact with a region formed from fleece of a chromatographic test strip, which contained a Δ⁹-THC-specific antibody in accordance with Example 3, and the test strip was incubated at room temperature for a period of 60 seconds.

After incubation with the sample of the analyte, the test strip was dipped in bidistilled water, which contained 1,4-dithioerythritol, commercially available with trade name Sputolysin® (from VWR), in four different concentrations, and the chromatography process was thus started. The completion of the test was reached within 5 to 10 minutes. The results of the four determinations are shown in Table 2:

| 1,4-dithioerythritol | |
|---|---|
| concentration | Test line signal |
| in the eluent (wt. %) | for Δ⁹-THC |
| 5% | Negative |
| 1% | Weak positive |
| 0.1% | Positive |
| 0% | Weak positive |

As can be seen from Table 2, mixing 1,4-dithioerythritol with the eluent in a concentration range of approximately 0.01 to approximately 1 wt. % leads to stronger signal development on the test line of the chromatographic test strip.

### Example 6

### Use of Sodium Hydroxide to Increase Sensitivity and Specificity

A region formed of fleece of a chromatographic test strip was impregnated with a solution of 2.0 wt. % bovine serum albumin (from Sigma Aldrich), 0.5 wt. % Brij (from Sigma Aldrich) and 0.03 wt. % sodium hydroxide in distilled water (fleece A). In parallel, so as to evaluate the effect of sodium hydroxide for the purposes of a chemical saliva preparation, a region formed of fleece of a second chromatographic test strip was impregnated with a solution of 2.0 wt. % bovine serum albumin and 0.5 wt. % Brij® in distilled water (fleece B).

Subsequently, pooled saliva (saliva mix from five people) was set to benzodiazepine concentrations of 20 ng/ml, 30 ng/ml, 50 ng/ml and 100 ng/ml, using a solution of benzodiazepine (from Cerilliant) diluted in methanol. After applying 10 μl of this doped pooled saliva to the wiping face of a standard wiping element (from Securetec Detektionssysteme), the wiping element was brought into contact with the impregnated region of the two chromatographic test strips, none of which contained a benzodiazepine-specific antibody, and the test strips were incubated at room temperature for a period of 30 seconds.

After incubation with the sample of the analyte, the test strips were dipped in bidistilled water, and the chromatography process was thus started. The completion of the test was reached in 5 to 10 minutes. The results of the determinations are shown in Table 3:

TABLE 3

| | Test line signal |
|---|---|
| Benzodiazepine | for benzodiazepine |
| concentration | Fleece A |
| 20 ng/ml | Weak positive |
| 30 ng/ml | Positive |
| 50 ng/ml | Positive |
| 100 ng/ml | Positive |

As can be seen from Table 3, impregnating the fleece with small amounts of sodium hydroxide leads to a stronger signal development on the test line of the chromatographic test strip and thus to an improvement in the benzodiazepine detection.

### Example 7

### Use of Fleece to Increase Sensitivity and Specificity

To evaluate the effect of different fleeces for the purposes of mechanical saliva preparation, chromatographic test strips were prepared, which comprised a region formed of Sontara® (from DuPont), Ahlstrom 8964 (from Ahlstrom), Rapid 24Q (from Whatman) and FS 2216 (from Freudenberg) respectively, for receiving a sample to be analysed.

Pooled saliva (saliva mix from five people) was set to cocaine concentrations of 5 ng/ml, 15 ng/ml, and 50 ng/ml using a solution of cocaine (from Cerilliant) diluted in methanol. After applying 10 μl of this doped pooled saliva to the wiping face of a standard wiping element (from Securetec Detektionssysteme), the wiping element was brought into contact with the region formed from fleece of each of the chromatographic test strips, none of which contained cocaine-specific antibody, and the test strips were incubated at room temperature for a period of 60 seconds.

After incubation with the sample of the analyte, the test strips were dipped in bidistilled water, and the chromatography process was thus started. The completion of the test was reached within 5 to 10 minutes. The results of the determinations are shown in Table 4:

TABLE 4

| | Test line signal | | |
|---|---|---|---|
| Cocaine | for cocaine | | |
| concentration | Sontara ® 8423 | Ahlstrom 8964 | Rapid 24Q |
| 5 ng/ml | Negative | Weak positive | Weak positive |
| 15 ng/ml | Weak positive | Positive | Positive |
| 50 ng/ml | Weak positive | Positive | Positive |

As can be seen from Table 4, using Ahlstrom 8964 fleece, Whatman Rapid 24Q fleece and Freudenberg FS 2216 fleece leads to a stronger signal development on the test line of the chromatographic test strip, and thus to an improvement in cocaine detection, already at a cocaine concentration of 5 ng/ml. This is basically because Ahlstrom® 8964, Whatman Rapid 24Q and Freudenberg FS 2216 mechanically hold back the solid and viscous saliva components respectively particularly well and thus exhibit better chromatographic properties.

### Example 8

### Effect of the Incubation of the Analyte and the Analyte-Specific Binding Partner on Sensitivity

In the context of a clinical trial, a saliva sample was consuming from methadone-taking test subjects, who had additionally taken Δ⁹-THC 8 hours at most before the sampling and had a very dry mouth without visible wet saliva, using a standard wiping element (from Securetec Detektionssysteme). Test subjects who had not taken Δ⁹-THC before sampling were used as a negative control. To determine the volume of saliva taken in each case, the wiping elements were weighed before and after the sampling.

To evaluate the effect of direct incubation of the analyte and the analyte-specific binding partner on the sensitivity of the detection method, the wiping elements in a first test series were each brought into contact with a region formed of fleece of a chromatographic test strip as disclosed in EP 0 699 906 A2 and EP 0 811 842 A2. Since the test strips disclosed in the above documents do not contain any analyte-specific binding partner in the region provided for receiving the sample, in both cases the analyte and the analyte-specific binding partner only can come into contact with one another after the start of the chromatography.

In a second test series, the wiping elements were each brought into contact with a region formed from fleece of a chromatographic test strip according to the invention, which contained a Δ⁹-THC-specific antibody in accordance with Example 3 in the region formed for receiving the sample, and the test strips were incubated at room temperature for a period of 60 seconds. Subsequently, each of the test strips was dipped in bidistilled water and the chromatography process was started. The completion of the test was reached within 5 to 10 minutes. The results are shown in Table 5:

TABLE 5

| Test | Saliva | | Test line signal |
|---|---|---|---|
| subject | volume | Test line signal | without |
| number | [μl] | with incubation | incubation |
| 1 | 2.1 | Positive | Positive |
| 2 | 1.4 | Positive | Negative |
| 3 | 1.0 | Weak positive | Negative |
| 4 | 1.5 | Positive | Weak positive |
| 5 | 0.8 | Positive | Negative |
| 6 | 1.1 | Negative | Negative |
| 7 | 1.2 | Weak positive | Negative |
| 8 | 1.4 | Positive | Negative |
| 9 | 1.5 | Weak positive | Negative |
| 10 | 2.0 | Positive | Positive |
| 11 (negative | 1.9 | Negative | Negative |
| control) | | | |
| 12 (negative | 1.4 | Negative | Negative |
| control) | | | |
| 13 (negative | 1.2 | Negative | Negative |
| control) | | | |

As can be seen from Table 5, even in test subjects with a very dry mouth (average sample volume in the positive controls: 1.4 μl; standard deviation: 0.4 μl), 60-second incubation of the analyte and the analyte-specific binding partner before the start of the chromatography process makes it possible to detect 3 times as many Δ⁹-THC-consumers as in conventional test systems which do not comprise incubation of the analyte and the analyte-specific binding partner before the start of the chromatography.

### Example 9

### Effect of the Incubation of the Analyte and the Analyte-Specific Binding Partner on the Sensitivity

Pooled saliva (saliva mix from five people) was initially set to a Δ⁹-THC concentration of 200 ng/ml using a solution of Δ⁹-THC (from Cerilliant) diluted in methanol. Subsequently, 8 μl of this doped pooled saliva was received using a standard wiping element (from Securetec Detektionssysteme).

Subsequently, the wiping element thus obtained was brought into contact with a region formed of fleece of a chromatographic test strip for a period of 10, 20, 40, 60, 100 and 180 seconds, in two test series, wherein in the first test series, chromatographic test strips, which contained a Δ⁹-THC-specific antibody in accordance with Example 3 in the region provided for receiving the sample, were used in each case.

In parallel, a second test series was carried out using chromatographic test strips, which did not contain the Δ⁹-THC-specific antibody in accordance with example 3 in the region formed for receiving the sample, but rather in a region spatially separated therefrom, in such a way that a contact between the analyte and the analyte-specific binding partner only occurred during the course of chromatography.

Subsequently, each of the test strips was dipped in bidistilled water and the chromatography process was started. The complete development of the test took 5 to 10 minutes. The results are shown in Table 6:

TABLE 6

| | Test line signal with | Test line signal without |
|---|---|---|
| Incubation | Δ⁹-THC-specific | Δ⁹-THC-specific |
| time | antibody in the | antibody in the |
| [sec] | receiving region | receiving region |
| 10 | ++ | + |
| 20 | ++ | + |
| 40 | ++ | (+) |
| 60 | ++ | (+) |
| 100 | ++ | ((+)) |
| 180 | ++ | ((+)) |

++ Strong positive signal
+ Positive signal
(+) Weak positive signal
((+)) Borderline positive signal—transition to negative signal

As can be seen from Table 6, in chromatographic test strips which contain the Δ⁹-THC-specific antibody in the region provided for receiving the sample, already after 10-second incubation of the analyte and the analyte-specific binding partner before the start of the chromatography, a strong signal development on the test line of the chromatographic test strip can be achieved. At the same time, it can be seen that the strength of the test line signal does not change with increasing incubation time.

The latter is on one hand attributed to the fact that the analyte forms a complex with the analyte-specific binding partner immediately after the wiping element and chromatographic test strip are brought into contact, and thus does not or only small part of it bind non-specifically to the fleece of the test strip. On the other hand, that there is also no strengthening of the test line signal with increasing incubation time because the relatively high analyte concentration of 200 ng/ml covers up the positive effect of a longer incubation time.

When the above test series are repeated using a g-THC concentration of 20 ng/ml, the results shown in Table 7 are obtained:

TABLE 7

| | Test line signal with | Test line signal without |
|---|---|---|
| Incubation | Δ⁹-THC-specific | Δ⁹-THC-specific |
| time | antibody in the | antibody in the |
| [sec] | receiving region | receiving region |
| 10 | ((+)) | − |
| 20 | (+) | − |
| 40 | (+) | − |
| 60 | + | − |
| 100 | ++ | − |
| 180 | ++ | − |

++ Strong positive signal
+ Positive signal
(+) Weak positive signal
((+)) Borderline positive signal—transition to negative signal
− Negative signal

As can be seen from Table 7, in the case of chromatographic test strips which contain the Δ⁹-THC-specific antibody in the region provided for receiving the sample, and thus make direct incubation of the analyte and the analyte-specific binding partner possible, Δ⁹-THC can be detected also at a concentration of merely 20 ng/ml, already after a short incubation time.

As the incubation time increases, the intensity of the test line signal increases, wherein the maximum test line signal is reached from an incubation time of 100 seconds. At the same time, it can be seen from Table 7 that in the case of the test variant without a Δ⁹-THC-specific antibody in the sample-receiving region of the chromatographic test strip, it is not possible to detect Δ⁹-THC at a concentration of 20 ng/ml.

1. Method for determining an analyte, comprising the steps of:
(a) providing a test element, comprising
(i) a first region which is provided for receiving the eluent,
(ii) a second region which is provided for applying a sample containing the analyte, and which comprises a binding partner specific to the analyte,
(iii) a third region which is provided for the visual detection of the analyte,
(iv) a fourth region which is provided for receiving the excess of eluent, and
(v) optionally a housing,
(b) receiving a sample containing the analyte from a sample surface, using a wiping element,
(c) bringing the wiping element into contact with the second region of the test element for a period of at least 10 seconds, wherein at least part of the sample is transferred from the wiping element to the test element and the incubation of analyte with the analyte-specific binding partner occurs,
(d) bringing the first region of the incubated test element into contact with an eluent, and
(e) determining the presence and/or the amount of the analyte,
wherein the test element and/or the wiping element comprises an analyte transfer reagent which contains at least one analyte-non-specific substance selected from the group consisting of a protein, a protein mixture, a carbohydrate and a sugar alcohol.

2. Method according to claim 1, characterised in that a chromatographic test strip is used as the test element.

3. Method according to either claim 1, characterised in that an antibody or a functional antibody fragment is used as the analyte-specific binding partner.

4. Method according to claim 1, characterised in that a wiping element having one or a plurality of wiping faces is used.

5. Method according to claim 1, characterised in that the wiping element and the test element are brought into contact with one another for a period of approximately 10 seconds to approximately 600 seconds, in particular approximately 60 seconds to approximately 180 seconds.

6. Method according to claim 1, characterised in that the test element comprises an analyte transfer reagent which contains at least one analyte-non-specific substance selected from the group consisting of a carbohydrate and a sugar alcohol.

7. Method according to claim 1, characterised in that the wiping element comprises an analyte transfer reagent which contains at least one analyte-non-specific substance selected from the group consisting of a protein and a protein mixture.

8. Method according to claim 1, characterised in that the second region of the test element further comprises at least one means for chemically and/or mechanically treating the sample containing the analyte.

9. Method according to claim 8, characterised in that the chemical sample treatment means comprises a substance selected from the group consisting of an acid, a base, a buffer, an organic solvent and a detergent.

10. Method according to claim 8, characterised in that the mechanical sample treatment means comprises a fabric and/or a fleece.

11. Method according to claim 1, characterised in that it comprises a combination of a competitive test format and a non-competitive test format.

12. Method according to claim 1, characterised in that the analyte is determined with a specificity of at least 95% and/or with a sensitivity of at least 90%.

13. Method according to claim 1, characterised in that a narcotic, in particular a substance selected from the group consisting of dissociatives, deliriants, empathogens, entactogens, hypnotics, narcotics, psychedelics, sedatives and stimulants, is determined.

14. Method according to claim 1, characterised in that a plurality of analytes, in particular 2 to 20 different analytes, are determined simultaneously.

15. Method according to claim 1, characterised in that a body fluid, in particular blood, urine or saliva, is used as the sample.

16. Method according to claim 1, characterised in that a sample is used having a volume of approximately 0.05 μl to approximately 20 μl, in particular having a volume of approximately 0.1 μl to approximately 5 μl.

17. Test element for determining an analyte, comprising:
(i) a first region which is provided for receiving the eluent,
(ii) a second region which is provided for applying a sample containing the analyte, and which comprises a binding partner specific to the analyte,
(iii) a third region which is provided for the visual detection of the analyte,
(iv) a fourth region which is provided for receiving the excess of eluent,
(v) optionally a housing, and
(vi) an analyte transfer reagent which contains at least one analyte-non-specific substance selected from the group consisting of a protein, a protein mixture, a carbohydrate and a sugar alcohol.

18. Use of a wiping element in a method according to claim 1 for receiving an analyte from a surface and for transferring the analyte on the test element, wherein the wiping element comprises an analyte transfer reagent containing at least one analyte-non-specific substance selected from the group consisting of a protein, a protein mixture, a carbohydrate and a sugar alcohol.

19. Kit for determining an analyte, comprising:
(a) a test element, comprising
(i) a first region which is provided for receiving the eluent,
(ii) a second region which is provided for applying a sample containing the analyte, and which comprises a binding partner specific to the analyte,
(iii) a third region which is provided for the visual detection of the analyte,
(iv) a fourth region which is provided for receiving the excess of eluent, and
(v) optionally a housing, and
(b) a wiping element provided for receiving a sample of the analyte, wherein
the test element and/or the wiping element comprise an analyte transfer reagent which contains at least one analyte-non-specific substance selected from the group consisting of a protein, a protein mixture, a carbohydrate and a sugar alcohol.
